(19) United States
(12) Patent Application Publication
(10) Pub. No.: US 2013/0165628 A1
(43) Pub. Date: Jun. 27, 2013

(54) Double Binding Constructs

(71) Applicant: Hendrik Mario Geysen, Charlottesville, VA (US)

(72) Inventor: Hendrik Mario Geysen, Charlottesville, VA (US)

(73) Assignee: SRI INTERNATIONAL, Menlo Park, CA (US)

(21) Appl. No.: 13/726,115

(22) Filed: Dec. 22, 2012

Related U.S. Application Data
(60) Provisional application No. 61/579,835, filed on Dec. 23, 2011.
(60) Provisional application No. 61/651,513, filed on May 24, 2012.

(51) Int. Cl. A61K 47/48

(52) U.S. Cl. CPC A61K 47/48246
USPC 530/324; 530/351; 530/300; 530/345

(57) ABSTRACT

The disclosure provides methods and materials for improving the pharmacokinetic properties of drugs. For example, a construct is provided having a drug covalently joined to first and second ligands, or a drug covalently joined to a first ligand and a molecular weight increasing moiety. The ligands have affinity for binding partners, and in a physiological fluid, an equilibrium forms between bound and free forms of the construct. The constructs retain most of the drug's activity while simultaneously increasing half life.

### CROSS REFERENCE TO RELATED APPLICATIONS

This application claims priority under 35 U.S.C. §119(e)(1) to No. 61/579,835, filed Dec. 23, 2011, and No. 61/651,513, filed May 24, 2012, the disclosures of which are incorporated herein by reference in their entireties.

### INTRODUCTION

Short half-life and a narrow therapeutic window are common pitfalls for protein, peptide, and small-molecule pharmaceuticals. Short half-life is typically a result of clearance of the pharmaceutical, such as clearance via the kidneys in mammals. Narrow therapeutic window is a problem when side effects limit the upper tolerable dose of a therapeutic, yet efficacy limitations impose a minimum dosage. To overcome short half-life, it is common to administer a large dose of the therapeutic, although a larger dose increases the likelihood of side effects, patient non-compliance, etc. Accordingly, overcoming the dual and competing problems of short half-life and a narrow therapeutic window remains a concern in the pharmaceutical industry.

Relevant art: EP0486525B1, PCT/US00/35325, PCT/US09/60721, Knudsen et al. “Potent Derivatives of Glucagon-like Peptide-1 with Pharmacokinetic Properties Suitable for Once Daily Administration” J. Med. Chem. 2000, 43, 1664-1669.

### SUMMARY OF THE INVENTION

We have developed a linking motif that addresses the problems of short half-life and narrow therapeutic window. An active component is coupled to two functional components. The first component (also called a “molecular weight increasing moiety”) is reversibly or permanently bound to the active component, and increases the molecular weight of the construct to reduce clearance of the active component, thereby addressing half-life limitations. The second component is reversibly bound to the active component and inactivates or reduces the activity of the active component upon binding. The inactivated, bound state therefore serves as a reservoir of the active moiety. The concept of a reservoir of inactivated compounds (i.e., active compound bound to two large circulating molecules) is not taught in the art.

In some embodiments of the inventive constructs, an active moiety has at least two binding moieties that are each capable of reversibly binding a large circulating molecule such as a protein. In some embodiments of the inventive constructs, an active moiety is covalently bound to a large component such as a protein or polymer, and further contains a binding moiety that is capable of reversibly binding a large circulating molecule such as a protein.

In one aspect, there is provided a construct comprising a drug covalently joined to first and second ligands having affinity for first and second binding partners, respectively, of a physiological fluid, wherein in the fluid an equilibrium is formed between (a) the construct in free form, which retains most of the drug's activity, and (b) the construct in a ternary complex with the binding partners, which retains less of the drug's activity.

In a second aspect, there is provided a construct comprising a drug covalently joined to a molecular weight increasing moiety that increases half life of the construct and a ligand having affinity for a binding partner of a physiological fluid, wherein in the fluid an equilibrium is formed between (a) the construct in free form which retains relatively more of the drug's activity, and (b) the construct in a binary complex with the binding partner, which retains relatively less of the drug's activity.

In a further aspect, there is provided a construct comprising a drug covalently joined to a ligand having affinity for a binding partner of a physiological fluid, wherein in the fluid an equilibrium is formed between (a) the construct in free form which retains relatively more of the drug's activity, and (b) the construct in a binary complex with the binding partner, which retains relatively less of the drug's activity, wherein the drug has a molecular weight of at least 10,000 Da.

In a further aspect, there is provided a method of modifying pharmacokinetics of a drug comprising the step of: incorporating the drug into a construct according to the above aspects.

In a further aspect, there is provided a method of administering a drug comprising the step of administering to an individual a construct according to the above aspects.

The above aspects, wherein the construct at least doubles the half-life of the drug in the fluid.

The above aspects, wherein the first and second binding partners are selected from human serum albumin (HSA), heat shock proteins (HSPs), Fc, ubiquitin, fibrinogens, immunoglobulins, α₁-antitrypsin, α₂-macroglobulins, transferrin, prothrombin, avidin, and streptavidin.

The above aspects, wherein the molecular weight increasing moiety is selected from synthetic polymers, proteins, polysaccharides, and glucoseaminoglycans.

The above aspects, wherein the first ligand is covalently attached to the drug via a first linker (L1) and wherein the second ligand is covalently attached to the drug via a second linker (L2).

The above aspects, wherein the drug is covalently joined to the ligand and the molecular weight increasing moiety through a linker (L3).

The above aspects, wherein the drug is covalently joined to the ligand and the molecular weight increasing moiety through a first linker (L1) a second linker (L2), respectively.

The above aspects, wherein the molecular weight increasing moiety has a molecular weight greater than 10,000 Da.

The above aspects, wherein the first and second binding partners are the same molecular species.

The above aspects, wherein the drug is further covalently joined to a third ligand having affinity for a third binding partner.

The above aspects, wherein the Kd of the construct is not more than about 10 times the Kd of the free drug.

The above aspects, wherein the ligands are selected from fatty acids, peptides, peptidomimetics, epitopes, antigens, metal ion containing complexes, pharmaceutically active moieties, and small organic moieties.

The invention specifically provides all combinations of the recited aspects, as if each had been laboriously individually set forth.

### BRIEF DESCRIPTION OF THE DRAWINGS

FIG. 1 depicts single binding of a drug having a ligand to a binding partner. The drug remains active while bound.

FIG. 2 depicts single binding of a drug having a ligand to a binding partner. The drug is inactive while bound.

FIG. 3 depicts double binding of a drug having two ligands to two binding partners. The drug is inactive while bound to the first binding partner.

FIG. 4 depicts double binding of a drug having two ligands to two binding partners. The drug is inactive while bound to the first binding partner.

FIG. 5 depicts double binding of a drug having two ligands to two binding partners. The drug remains active while bound to the first binding partner but is inactive when bound to the second binding partner.

FIG. 6 depicts single binding of a construct having a ligand and a molecular weight increasing moiety, wherein the latter is bound to a drug via a covalent linkage. The drug is inactive when bound to the binding partner.

FIGS. 7A and 7B schematically depict in vivo concentrations for a drug having a single ligand (and no molecular weight increasing moiety) and for a drug having two ligands.

FIG. 8 provides calculated values for: in vivo concentrations for a drug having a single ligand and no molecular weight increasing moiety (8A); in vivo concentrations for a drug having two ligands (8B); and % activity for a drug having two ligands (8C).

FIGS. 9A and 9B provide example constructs prepared according to the invention.

### DETAILED DESCRIPTION OF PARTICULAR EMBODIMENTS

In one aspect the invention provides a construct comprising a drug covalently joined to first and second ligands having affinity for first and second binding partners, respectively, of a physiological fluid, wherein in the fluid an equilibrium is formed between (a) the construct in free form, which retains most of the drug's activity, and (b) the construct in a ternary complex with the binding partners, which retains less of the drug's activity.

In another aspect the invention provides a construct comprising a drug covalently joined to a molecular weight increasing moiety that reduces kidney clearance of the construct and a ligand having affinity for a binding partner of a physiological fluid, wherein in the fluid an equilibrium is formed between (a) the construct in free form which retains relatively more of the drug's activity, and (b) the construct in a binary complex with the binding partner, which retains relatively less of the drug's activity.

In another aspect there is provided a construct comprising a drug covalently joined to a ligand having affinity for a binding partner of a physiological fluid, wherein in the fluid an equilibrium is formed between (a) the construct in free form which retains relatively more of the drug's activity, and (b) the construct in a binary complex with the binding partner, which retains relatively less of the drug's activity, wherein the drug has a molecular weight of at least 10,000 Da. In this aspect, the drug moiety is large enough (i.e., is a macromolecule) to provide a desirable half-life.

The drug or pharmaceutically active moiety incorporated into the subject constructs may be small organic moieties, peptides, proteins, or polynucleotides, and may be naturally occurring or synthetic. Examples of drugs include hormones, antibacterials, interferon, antibodies and antibody fragments (e.g., an antibody fragment comprising a Fab or a F(ab′)₂), blood clotting factors, G-CSF, GLP-1 agonists, aviptadil, bivalirudin, calcitonin, carperitide, desmopressin, enfurivirtide, eptifibatide, exenatide, lanreotide, liraglutide, mifamurtide, nesirtide, pramlintide, romiplostim, taltirelin, teriparatide, dulaglutide, albiglutide, lixinsenatide, taspoglutide, iodipamide, oxyphenbutazone, propofol, diflunisal, diazepam, halothane, ibuprofen, indoxyl sulphate, CMPF, azapropazone, indomethacin, PTH, TIB, DIS, warfarin, indoxyl sulphate, and the like. Where such drugs are peptides, the subject constructs may include the entire peptide or a pharmaceutically active subset of the peptide. Where the drug is a macromolecule, the drug moiety is a protein, or pegylated peptide, or pegylated small molecule, or the like (further examples of macromolecular drugs are known in the art).

Drug activity is typically measured by Kd; hence, a convenient way to confirm that the construct retains most of the drug's activity is to show that the Kd of the construct is no more than about 10 times the Kd of the unmodified drug (i.e., the drug not joined to first and second ligands). For example, in some embodiments the Kd of the construct comprising first and second ligands is less than about 9, 8, 7, 6, 5, 4, 3, 2, 1.5, 1.25, or 1.1 times the Kd of the unmodified drug. Methods for measuring Kd values are commonly known in the art, and generally involve preparing a dose response curve.

The first and second ligands of the subject constructs selectively bind to binding partners. In some embodiments the binding partners are circulating molecules (i.e., compounds found circulating in a patient, such as in the blood or lymph), examples of which include proteins, lipoproteins, enzymes, polynucleotides, and the like. Specific examples include human serum albumin (HSA), heat shock proteins (HSPs), Fc, ubiquitin, fibrinogens, immunoglobulins, α₁-antitrypsin, α₂-macroglobulins, transferrin, prothrombin, avidin, streptavidin, and the like. In some embodiments the two binding partners are the same (e.g., two HSA molecules), and in other embodiments, the two binding partners are different (e.g., an HSA molecule and avidin).

Ligands suitable for the subject constructs are moieties capable of binding to the binding partners described herein, and the ligands are typically selected based on the desired binding partner for a particular application. Examples of ligands include fatty acids, peptides, peptidomimetics, epitopes, antigens, metal ion containing complexes, pharmaceutically active moieties, small organic moieties, and the like. Pharmaceutically active moieties are suitable as ligands because many such compounds bind to binding partners as described herein. Examples of fatty acids include those capable of binding HSA, such as lauric acid, myristic acid, palmitic acid, stearic acid, oleaic acid, diacids such as undecanedioic acid, unsaturated fatty acids, and the like. For example, a lysine residue in a drug can be modified to include a fatty acid side chain suitable for binding to a binding partner. See Curry et al., “Fatty Acid Binding . . . ” Biochimica et Biophysica Acta 1441 (1999) 131-140 as well as Knudsen et al., “Potent derivatives . . . ” J. Med. Chem., 43, (2000) 1664-1669), the contents of which are incorporated by reference. Examples of small organic molecules include biotin, ATP/ADP, 4-(p-iodophenyl)-butanoic acid and derivatives thereof, ibuprofen, diazepam, warfarin, CMPF, azapropazone, iodipamide, oxyphenbutazone, phenylbutazone, 3-indoxyl sulfate, diflunisal, 3,5-diiodosalicylic acid, indomethacin, and the like. Examples of peptides include Fc regions, polyhistidine tags, albumin affinity peptides, and synthetic peptides such as those described herein.

In a physiological fluid (e.g., whole blood, plasma, cerebrospinal fluid, lymph fluid, etc.) of an animal (e.g., human), binding of the first binding partner to a construct forms a binary complex. The increase in mass of the construct due to the first binding partner results in an increase in the half life of the construct, wherein the mass increase may be sufficient to decrease the rate of clearance of the construct (e.g., clearance by kidneys). In some embodiments, the half life of the construct is increased (relative to the drug without the ligand and therefore without the first binding partner) by 10%, 25%, 75%, or 100%, or by a factor of greater than or equal to 3, 4, 5, 6, 7, 8, 9, 10, 15, 20, 25, 50, or 100 compared with the construct without the first binding partner. The binary complex retains most of the activity of the unmodified drug, and also retains most of the activity of the construct in free form. For example, the Kd value of the binary complex is less than about 9, 8, 7, 6, 5, 4, 3, 2, 1.5, 1.25, or 1.1 times the Kd of the unmodified drug. The term “half life” as used herein refers to the total amount of a subject construct present in the physiological fluid (in all of the various forms of the construct, such as the free form and the bound form or forms).

Binding of the second binding moiety to a binary complex forms a tertiary complex. The tertiary complex retains the increased half life of the binary complex (and, in fact, may have a half life that is even greater than that of the binary complex), but retains less activity compared with the binary complex, the construct in free form, and the unmodified drug. The pharmacological activity of the tertiary complex is, in some embodiments, less than about 50%, 25%, 10%, 5%, 3%, 1%, 0.5%, or 0.1% of the binary complex as measured, for example, by Kd values. For example, the Kd of the tertiary complex may be greater than 2, 3, 4, 5, 6, 7, 8, 9, 10, 20, 25, 50, or 100 times that of the binary complex.

The inventive constructs are not limited to one or two ligands. In some embodiments, the subject constructs contain a third ligand that binds a third binding partner.

The molecular weight increasing moiety (also “MWIM”) serves a function similar to the first ligand and first binding partner as described herein—i.e., the molecular weight increasing moiety provides sufficient mass to the construct to increase the construct's half life. Thus, in some embodiments, rather than having first and second ligands, a construct has a first ligand and a molecular weight increasing moiety. The mass of the molecular weight increasing moiety is sufficient to increase the half life of the construct, such as by decreasing the rate of clearance of the construct (e.g., clearance by kidneys). In some embodiments, the half life of the construct is increased (relative to the drug without the molecular weight increasing moiety) by 10%, 25%, 75%, or 100%, or by a factor of 3, 4, 5, 6, 7, 8, 9, 10, or more than 10.

In some embodiments, constructs having a molecular weight increasing moiety have only one ligand that is covalently attached to the drug via a linking moiety and that is for binding to a binding partner. In a physiological fluid, binding of a construct having a molecular weight increasing moiety to a binding partner forms a binary complex (which may also be referred to herein as a “binary complex having a molecular weight increasing moiety” so as to distinguish it from a binary complex having two ligands but only one ligand bound to a binding partner). Such a binary complex retains the increased half life of the construct, but retains less activity compared with the construct in free form or the unmodified drug. The pharmacological activity of such a binary complex is, in some embodiments, less than about 50%, 25%, 10%, 5%, 3%, 1%, 0.5%, or 0.1% of the construct in free form as measured, for example, by Kd values. For example, the Kd of such a binary complex may be greater than 2, 3, 4, 5, 6, 7, 8, 9, 10, 20, 25, 50, or 100 times that of the construct in free form.

It is preferred that the molecular weight increasing moiety causes minimal reduction of the pharmaceutical activity of the drug. For example, the Kd value of a construct having a ligand and a molecular weight increasing moiety is less than about 9, 8, 7, 6, 5, 4, 3, 2, 1.5, 1.25, or 1.1 times the Kd of the unmodified drug.

Examples of molecular weight increasing moieties include large molecules such as proteins (e.g., HSA, etc.), polysaccharides (e.g., CMC, hyaluronic acid, etc.), synthetic polymers (e.g., PEG, polylactic acid, etc., including copolymers), glucoseaminoglycans (e.g., heparin), and the like. The molecular weight of suitable molecular weight increasing moieties may be greater than 5, 10, 15, 20, 25, 30, 35, 40, 45, or 50 kDa.

In some embodiments, the molecular weight increasing moiety is provided as two or more separate entities that, in combination, have the effects described herein (e.g., increase half life of the construct and minimally reduce pharmacological activity). For example, two synthetic polymers such as PEG polymers can be attached to the drug at separate locations or attached to a linker that is attached to the drug, wherein each PEG polymer is individually too small to significantly increase half life of the drug but the combination of the two PEG polymers is sufficiently large to increase half life of the drug.

In some embodiments, the MWIM increases the half-life of the construct by a factor of equal to or greater than 3, 4, 5, 10, 15, 20, 25, 50, or 100 compared with the half-life of the construct without the MWIM. For example, a control construct lacking the MWIM has a half-life of 1 hour, wherein the construct with the MWIM has a half life of at least 2, 3, 5, 10, 15, or 25 hours.

In some embodiments the binding constant, Kd, for each binding event of the subject constructs is less than or equal to about 10 μM, 5 μM, 1 μM, or 500 nM. The Kd for binding of the first binding partner with the first ligand and the Kd for binding of the second binding partner with the second ligand may be the same or different, and will depend on the identities of the ligands and binding partners. The Kd can be selected and tuned to a desired value for an intended application. For example, as shown herein, altering amino acid sequences can produce ligands that have stronger or weaker affinities for a binding partner.

As an example, a construct can be prepared having a first ligand with a high affinity for a first binding partner (e.g., about 1 μM for HSA), and a second ligand with a very high affinity for a second binding partner (e.g., about 500 nM for HSA). When administered to a subject, such a construct would form an equilibrium between the free form, the binary complex, and the tertiary complex. Because of the selected binding affinities, the equilibrium would lie heavily toward the tertiary complex. Most of the construct will in the form of the binary or tertiary complex, such that the half life of the construct will be elevated compared with the construct in free form. Furthermore, because the tertiary complex has reduced activity, but the binary complex retains most of the activity of the free form, a larger dose can be provided to the subject without observing the side effects typical of a similar large dose of the unmodified drug. The tertiary complex is a less active form of the drug and acts as a non-harmful (i.e., not causing side effects) in vivo reservoir of the more active binary complex.

Similarly, a construct can be prepared having a first ligand with a high affinity for a first binding partner and a molecular weight increasing moiety. When administered to a subject, such a construct would form an equilibrium between the free form and the binary complex, both of which have an increased half life compared with the unmodified drug due to the presence of the molecular weight increasing moiety. Furthermore, most of the construct will be present in the form of the binary complex having reduced activity. Again, a larger dose can be delivered to the subject without observing the side effects typical of a similar large dose of the unmodified drug.

### Structure

A drug in a subject construct is covalently bound to the first ligand. The drug is also covalently bound to a second ligand or molecular weight increasing moiety. Such covalent bonding is via linker moieties. In some embodiments, the linker moieties directly link the drug with the first ligand, second ligand, or molecular weight increasing moiety. In other embodiments, two or more linker moieties are indirectly connected to the drug, such as via a branch moiety.

For example, various schemes are possible for connecting a first ligand and second ligand (or molecular weight increasing moiety) to a drug. In a first scheme, a first linker moiety directly connects the first ligand with the drug, and a second linker moiety directly connects the second ligand (or molecular weight increasing moiety) with the drug. In such embodiments, both ligands are directly connected to the drug. This scheme is represented by ligand-linker-drug-linker-ligand and ligand-linker-drug-linker-MWIM.

In a second scheme, the first ligand and first linker moiety are connected to a branch moiety, and the second ligand (or molecular weight increasing moiety) and second linker moiety are also connected to the branch moiety. The branch moiety is then directly connected to the drug via a third linker (i.e., a covalent bond, amino acid, peptide, etc. as defined herein for linkers generally). In such embodiments, neither ligand is directly connected to the drug. This scheme is represented by ligand-linker-branch (-linker-drug)-linker-ligand or ligand-linker-branch(-linker-drug)-linker-MWIM.

In a third scheme, the first ligand and the second ligand (or molecular weight increasing moiety) are connected to each other in serial via a first linker moiety. The combined structure is then directly connected to the drug via a second linker. In such embodiments, only one of the ligands is directly connected to the drug. This scheme is represented by ligand-linker-ligand-linker-drug or ligand-linker-MWIM-linker-drug or MWIM-linker-ligand-linker-drug.

Examples of linker moieties include a bond (e.g., a direct covalent bond, a peptide bond, etc., such that the ligand or molecular weight increasing moiety is directly attached to the drug), an amino acid (e.g., Glu, etc.), a peptide, a heteroatom (e.g., —S—, —O—, —N—, etc.), C₁-C₂₄ alkylene (including cycloalkylene, unsaturated alkylene, substituted alkylene, heteroatom-containing alkylene, and the like, such as methylene, ethylene, propylene, etc.), C₅-C₂₄ arylene (including substituted arylene, heteroarylene, and the like, such as phenylene, etc.), and other moieties that have a minimum of two functional groups, as well as combinations thereof (e.g., alkarylene, aralkylene, an amino acid with an alkylene, etc.).

Examples of branch moieties include amino acids having a sidechain (e.g., cysteine, etc.), peptides having at least one amino acid with a sidechain, branched alkyl, aryl, and other moieties that have a minimum of three functional groups.

Examples of suitable functional groups include halo, hydroxyl, sulfhydryl, C₁-₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO—), carbamoyl (—(CO)—NH₂), mono-substituted C₁-C₂₄ alkylcarbamoyl (—(CO)—NH(C₁-C₂₄ alkyl)), di-substituted alkylcarbamoyl (—(CO)—N(C₁-C₂₄ alkyl)₂), mono-substituted arylcarbamoyl (—(CO)—NH-aryl), thiocarbamoyl (—(CS)—NH₂), carbamido (—NH—(CO)—NH₂), cyano (—C≡EN), isocyano (—N+≡C—), cyanato (—O—CEN), isocyanato (—O—N≡C—), isothiocyanato (—S—C≡N), azido (—N═N+═N—), formyl (—(CO)—H), thioformyl (—(CS)—H), amino (—NH₂), mono- and di-(C₁-C₂₄ alkyl)-substituted amino, mono- and di-(C₅-C₂₀ aryl)-substituted amino, C₂-C₂₄ alkylamido (—N—(CO)-alkyl), C₅-C₂₀ arylamido (—NH—(CO)-aryl), imino (—CR═NH where R =hydrogen, C₁-C₂₄ alkyl, C₅-C₂₀ aryl, C₆-C₂₀ alkaryl, C₆-C₂₀ aralkyl, etc.), alkylimino (—CR═N(alkyl), where R=hydrogen, alkyl, aryl, alkaryl, etc.), arylimino (—CR═N(aryl), where R=hydrogen, alkyl, aryl, alkaryl, etc.), nitro (—NO₂), nitroso (—NO), sulfo (—SO₂—OH), sulfonato (—SO₂—O—), C₁-C₂₄ alkylsulfanyl (—S-alkyl; also termed “alkylthio”), arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O—)₂), phosphinato (—P(O)(O—)), phospho (—PO₂), and phosphino (—PH₂), mono- and di-(C₁-C₂₄ alkyl)-substituted phosphino, and mono- and di-(C₅-C₂₀ aryl)-substituted phosphino.

Linkers are bound to the drug in the subject constructs at any suitable binding location. For example, where the drug is a peptide or protein, the linkers may be bound to the drug at the N-terminus or C-terminus. Alternatively, the linker may be bound to the drug via a sidechain present on one of the amino acids of the drug. For example, a linker may be bound to a cysteine residue in a drug via the sulfur of the cysteine side chain. Such binding motifs also apply for ligands and molecular weight increasing moieties when the linking moiety is a direct covalent bond.

### Preparation

The subject constructs can be formed using any suitable method of synthesis. Examples include gene expression and preparative synthetic reactions and linkage reactions.

For example, the unmodified form of the drug can be synthetically modified to include one or more linkers, one or more ligands, and an optional molecular weight increasing moiety. Each of the components (linkers, ligands, and molecular weight increasing moiety) can be added in a separate synthetic step, or multiple modifications can be made in a single synthetic step where appropriate. For example, conjugation can be carried out at free thiol groups such as those present in lysine residues. This method is suitable for components of the subject constructs that are amino acids as well as those that are not amino acids.

Also for example, the construct can be prepared using expression of recombinant DNA (i.e., genetic fusion). Thus, polynucleotides coding for the drug, linker(s), ligand(s), and/or molecular weight increasing moiety can be constructed and then expressed in order to form the subject constructs in a single step. This method limits the construct components to amino acids, but has advantages such as defined structure and minimal post-manufacturing costs (e.g., purification, etc.). Fusion partners can be attached at either C or N termini of a drug of interest.

Combinations of the above methods can be used. Thus, for example, a moiety comprising the drug and attached linkers can be prepared using recombinant DNA expression, and the ligands can then be attached using synthetic reactions.

### Use

The subject constructs are useful for administering a drug to a subject. As used herein, “subject” includes animals such as humans, domesticated animals, and livestock. A subject construct is useful in treating conditions that are normally treated by the drug incorporated into the construct. Such conditions include microbial infections, diabetes, chronic pain, neurological disorders (e.g., Alzheimer's disease), etc.

In some embodiments preparation of a subject construct results in modifying the pharmacokinetics of a drug. Examples of pharmacokinetic properties that can be modified by such incorporation include drug half-life, hepatic first-pass metabolism, volume of distribution, degree of binding to a blood serum protein (e.g. albumin), degree of tissue targeting, cell type targeting, and the like.

In some embodiments, the subject constructs have an increased half life and simultaneously have a larger therapeutic window compared with the unmodified drug, and can therefore be administered in larger, less frequent doses. For example, the subject construct can be administered daily, or once every 2, 3, 4, 5, or 6 days, or once every 1, 2, 3, or 4 weeks, or 2 mo, 3 mo, or 6 mo. Such administration can be less than or equal to 50%, 25%, 15%, 10%, or 5% of the frequency required for the unmodified drug. In some embodiments, large dosages (i.e., dosages sufficient to allow for less frequent administration compared with normal dosages) of the unmodified drug are not desirable because of unwanted side effects; thus, therapeutic window limitations require frequent dosages. In the subject constructs, because of the equilibrium that is obtained in a physiological fluid between the free construct, the construct bound to one binding partner, and the construct bound to two binding partner (where two ligands are present), the amount of circulating active drug is reduced compared with the amount administered, and side effects are not observed even at significantly higher dosages. In other words, the maximum tolerated dose of the subject constructs is increased compared with that of the unmodified drug. In some embodiments, the maximum tolerated dose of the subject constructs is more than 10%, 25%, 50%, 75%, or 100% greater, or greater by a factor of 2, 3, 4, 5, or more than 5 compared with the maximum tolerated dose of the unmodified drug.

The subject constructs may be administered to a subject in any appropriate dosage, such as paternal, oral, transdermal, transmucosal (including rectal and vaginal), sublingual, by inhalation, or via an implanted reservoir in a dosage form. The term “parenteral” as used herein is intended to include, for example, subcutaneous, intravenous, and intramuscular injection.

Pharmaceutical formulations can be prepared using the subject constructs and one or more additional ingredients such as a pharmaceutically acceptable carrier. Generally in such formulations, the subject construct is contained in a “therapeutically effective” amount, i.e., in an amount effective to achieve its intended purpose. Determination of a therapeutically effective amount for any particular subject construct is within the capability of those skilled in the art. For example, a therapeutically effective dose can be estimated initially from cell culture assays. Also for example, a dose can be formulated to achieve a circulating concentration range that includes a Kd value as determined in cell culture. Such information can be used to more accurately determine useful doses in humans.

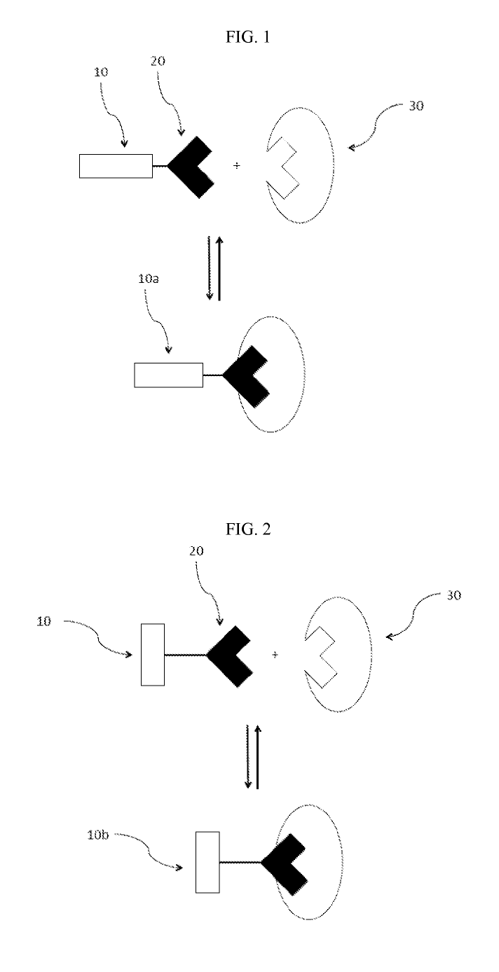

FIG. 1 illustrates the strategy of increasing half-life by non-covalent attachment to a large circulating molecule; for example the target is serum albumin. The bound and free states are pharmaceutically active, but the bound state has significantly reduced clearance. In this case the concentration of circulating biologically active component is the sum of both the free and bound states with clearance dictated by the free concentration. Ideally this requires low Kd values for the equilibrium as the lower the free concentration the longer the effective half-life. A complication with this strategy is that longer half-life is associated with higher biologically active concentrations often resulting in unwanted side-effects, i.e. a poor therapeutic window. This strategy is essentially equivalent to covalent attachment, either by chemical ligation or addition at the expression level. In FIG. 1, active hormone 10 is bound to ligand 20. HSA 30 binds with the construct and hormone 10a remains active.

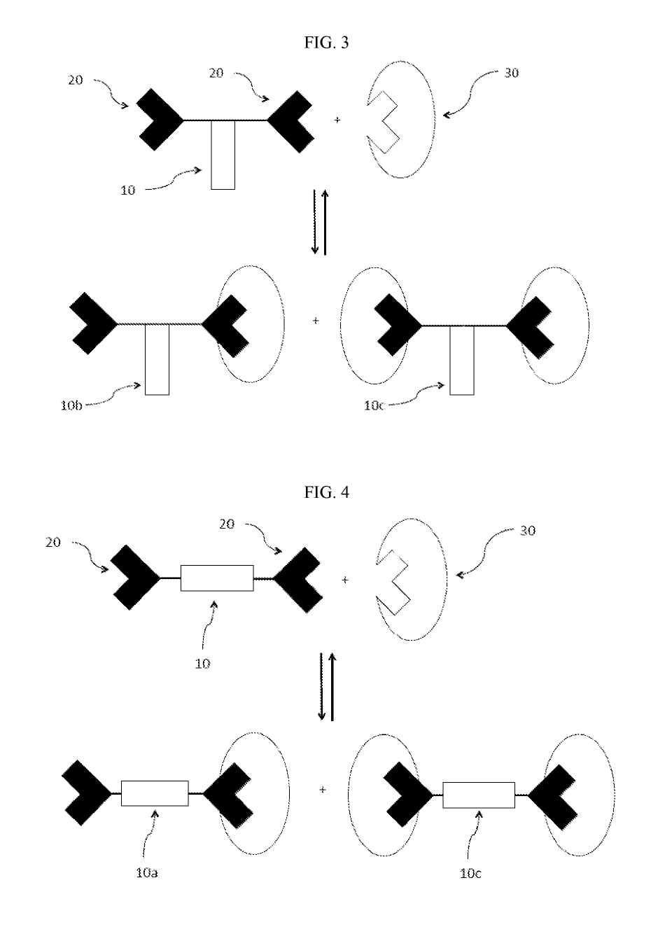

In FIG. 2, only the free or unbound state is biologically active and therefore by correct choice of Kd for the equilibrium it is possible to independently control both half-life and starting concentration of free construct. There can be, however, difficulty in obtaining the desired Kd for the equilibrium to achieve a selected half-life and starting active concentration. In FIG. 2, active hormone 10 is bound to ligand 20. HSA 30 binds with the construct and hormone 10b is inactive.

In FIG. 3, a bivalent approach results in much greater control of both the half-life and initial concentration of free, active compound. In particular the Kd values required, when the target carrier is serum albumin, are more easily achieved and significantly lower than would be required for the constructs in FIG. 1 or 2. In the schematic shown the bivalent albumin binding construct is attached to the ligand at only one site—thus the first and second ligands are connected via a linking moiety that directly connects to the drug (optionally via a linker that is not labeled in the figure). Binding of the first HSA to the construct results in an inactivated drug having reduced clearance. Binding of the second HSA to the construct further reduces activity and clearance. In FIG. 3, active hormone 10 is bound to ligands 20. HSA 30 binds with the construct and hormones 10b and 10c are inactive.

The depiction in FIG. 4 is similar to FIG. 3 with the variation that each carrier molecule (ligand) binding site is attached to the biologically active component at independent sites. The two ligands are each attached directly to the drug (optionally via linkers, which are not labeled in the figure). In FIG. 4, active hormone 10 is bound to ligands 20. HSA 30 binds with the construct and hormones 10b and 10c are inactive.

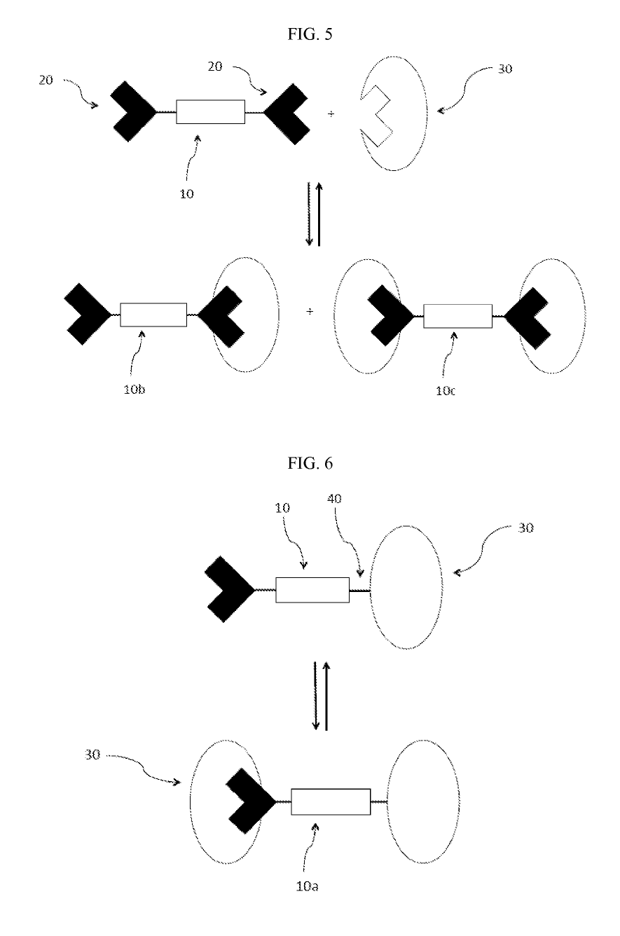

FIG. 5 represents an improvement over FIG. 4 because the singly-bound construct retains most of the activity of the unbound drug. The two ligands are each attached directly to the drug (optionally via linkers, which are not labeled in the figure). The doubly-bound construct is inactivated and represents a reservoir of drug. By tailoring the Kd of the two binding events, the half-life of the construct is increased without suffering the increase in side effects observed with the construct of FIG. 1. Thus, the therapeutic window is increased. In FIG. 5, active hormone 10 is bound to ligands 20. HSA 30 binds with the construct and hormone 10b remains active. Hormone 10c is inactive.

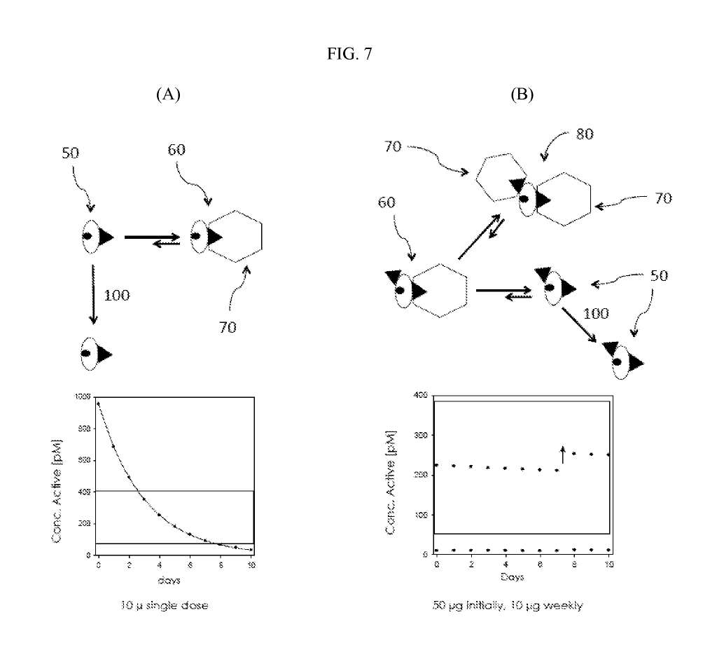

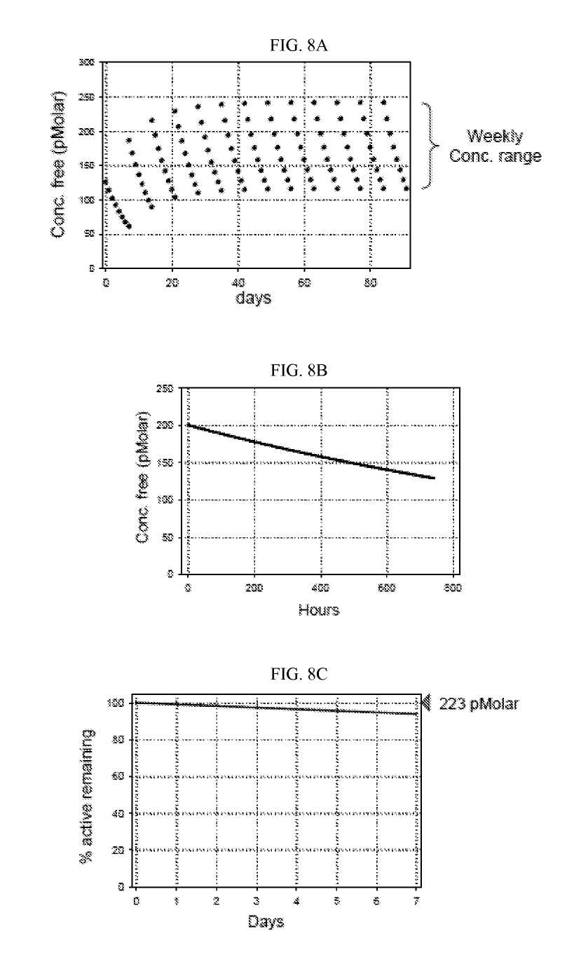

FIG. 6 represents a subject construct having a single ligand and a molecular weight increasing moiety (i.e., HSA). The molecular weight increasing moiety is covalently bound to the drug, and causes the drug to have a longer half life. The ligand reversibly binds an HSA moiety that causes a decrease in activity of the construct. Thus, by tailoring the Kd of the binding event, the activity and half life of the construct can be optimized. In FIG. 6, active hormone 10 is bound to ligand 20 and HSA 30, the latter via covalent bond 40. Upon binding second HSA 30, hormone 10a is inactive.

In FIG. 7A, a construct according to FIG. 1 is illustrated and idealized pharmacokinetics for the construct is provided. Renal clearance (labeled 100) occurs for unbound drug 50, but not for bound construct 60, which consists of drug 50 bound to binding partner 70. The construct has a single binding site for HSA with a Kd of 2.8E-5 M. A 10 μpg single dose is administered and observed over 10 days. The concentration of the construct falls drastically over the initial period of time after administration. The shaded box represents the therapeutically desirable in vivo concentrations of the drug. The drug is present in such concentrations for only a portion of the time displace. In contrast, FIG. 7B shows idealized pharmacokinetics for a construct according to FIG. 5 or 6. The construct has two binding sites, each for binding HSA with a Kd of 2.8E-5 M. A larger (50 μg) dose is administered, followed by a 10 μg dose after 1 week. The equilibrium of active free construct, active singly bound construct, and inactive doubly bound construct provides a concentration of active drug that remains within the shaded box (i.e., the therapeutically desirable concentration) for the entire period measured. In FIG. 7b, singly bound construct 60 is in equilibrium with unbound drug 50 (which can be cleared via renal clearance 100) and doubly bound inactive construct 80, which consists of drug bound to two binding partners 70.

In FIG. 8A, the concentration of free construct is provided for a construct according to FIG. 1 administered in single 2.5 mg weekly doses. The construct has a single binding site with a Kd of 3.2E-6. In FIG. 8B, a single 1.0 mg dose is tracked over 800 hours for a 2-binding site construct with Kd=2.8E-5 M. In FIG. 8C, the percentage of active component remaining for a single 50 μg dose is traced over seven days for a 2-binding site construct with Kd=2.8E-5 M.

### EXAMPLES

### Example 1

Peptide Synthesis: Peptide were synthesized by either manual or automated (ACT 496) solid phase synthesis, using Fmoc strategy on Rink Amide-polystyrene resin, substitution 0.7 mmol/g, using DIC/HOBt as activating agent (H. Rink, 1987, Tetrahedron Lett., 28, 3787; M. Bodansky 1984, in: Principles of Peptide Synthesis, Springer, Berlin: G. Field, R. Noble, 1990, int j pept protein res, 35, 161). Peptides were cleaved from the resin and side chain protecting groups were removed using a 25% Dichloromethane, 10% triisoprylsilane and 65% trifluoroacetic acid mixture. Disulfide bond formation was achieved by reacting of 1 eq iodine with the linear peptide in solution (a 10% trifluoroacetic acid, 90% methanol solution). Peptides were purified by reversed phase HPLC using a C18 stationary phase and a gradient of water/acetonitrile (0.1% trifluoroacetic acid). Identity and purity of the peptides were verified by LC-MS.

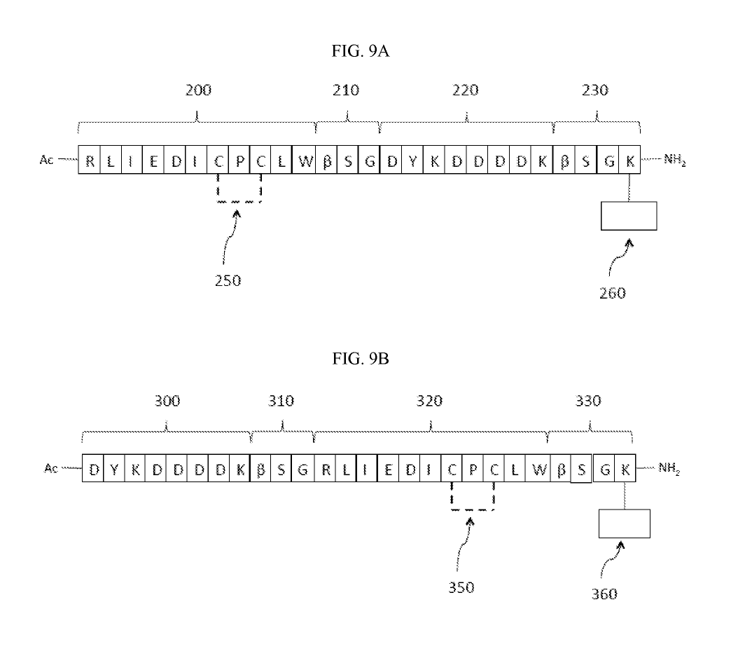

Using the above procedure, constructs were prepared having the structures shown in FIG. 9A and FIG. 9B. In FIG. 9A, disulfide bridge 250 is present within ligand (i.e., A1) 200, which is separated from drug moiety 220 by first linker (i.e., L1) 210. Second linker (i.e., L2) 230 separates drug 220 from a biotin moiety (i.e., A2) 260. The symbol β represents beta Alanine. In FIG. 9B, disulfide bridge 350 is present in ligand (i.e., A1) 320, which is present between first linker (i.e., L1) 310 and second linker (i.e., L2) 330. Second linker 330 connects to biotin moiety (i.e., A2) 360. First linker 310 links to drug 300. Again, β represents beta Alanine.

### Example 2

Constructs are prepared having the structures below. In such structures, “(aa)ₓ” represents an amino acid sequence of length x, wherein the x amino acids may be the same or different. Also, “HSA ligand” refers to a ligand as described herein that binds to HSA.

Palmityl-(aa)₃-GLP-1 agonist-(aa)₃-HSA ligand

Palmityl-(aa)₃-GLP-1 agonist-(aa)₃-Polyethylene glycol

ATP/ADP-(aa)₁-cysteine(alkylene-interferon)-(aa)₁polyhistidine tag

ATP/ADP-(aa)₁-cysteine(alkylene-interferon)-(aa)₁-HSA

Unless otherwise indicated, the disclosure is not limited to specific procedures, materials, or the like, as such may vary. It is also to be understood that the terminology used herein is for the purpose of describing particular embodiments only and is not intended to be limiting.

### Example 3

26-amino acid sequences were prepared, each comprising an hSA binder portion (11 amino acids and comprising a cysteine-proline-cysteine sequence capable of forming a disulfide bridge and a turn in the molecule), a first spacer (3 amino acids), a flag portion (8 amino acids), and a second spacer (4 amino acids). The second spacer terminated in an amino group and also comprised a covalently attached biotin moiety. At the other terminus, an acetyl group was present. Three different sequences were prepared, with the final amino acid at the acetyl-terminus varied between alanine (the control compound), arginine (construct 1), and lysine (construct 2). In viv experimentation showed a half life of 0.16 hours for the control, 34 hours for construct 1, and 53 hours for construct 2.

### Example 4

Double binding with inactivation was confirmed using a drug having two binding moieties covalently attached. One binding moiety reversibly bound biotin, the second binding moiety reversibly bound hSA. The drug was a Flag epitope and the receptor for testing the activity was Flag Ab.

### Example 5

Renin substrate stabilization was measured by preparation of a compound comprising: a covalently attached FRET pair; two separate hSA binder moieties adjacent to two separate cysteine-proline-cysteine sequences each capable of forming a disulfide bridge and a turn in the molecule; and the renin substrate. Cleavage inhibition by hSA binding was quantified using FRET measurements (at various hSA concentrations) and hSA concentrations.

As used in the specification and the appended claims, the singular forms “a,” “an,” and the include plural referents unless the context clearly dictates otherwise. Thus, for example, reference to “a reactant” includes not only a single reactant but also a combination or mixture of two or more different reactant.

It is to be understood that while the invention has been described in conjunction with the preferred specific embodiments thereof, the description above as well as the examples that follow are intended to illustrate and not limit the scope of the invention. Other aspects, advantages and modifications within the scope of the invention will be apparent to those skilled in the art to which the invention pertains.

1. A construct comprising a drug covalently joined to first and second ligands having affinity for first and second binding partners, respectively, of a physiological fluid, wherein in the fluid an equilibrium is formed between (a) the construct in free form, which retains most of the drug's activity, and (b) the construct in a ternary complex with the binding partners, which retains less of the drug's activity.

2. A construct comprising a drug covalently joined to a molecular weight increasing moiety that increases half-life of the construct and a ligand having affinity for a binding partner of a physiological fluid, wherein in the fluid an equilibrium is formed between (a) the construct in free form which retains relatively more of the drug's activity, and (b) the construct in a binary complex with the binding partner, which retains relatively less of the drug's activity.

3. The construct of claim 1, wherein the construct at least doubles the half-life of the drug in the fluid.

4. The construct of claim 1, wherein the first and second binding partners are selected from human serum albumin (HSA), heat shock proteins (HSPs), Fc, ubiquitin, fibrinogens, immunoglobulins, α₁-antitrypsin, α₂-macroglobulins, transferrin, and prothrombin.

5. The construct of claim 1, wherein the first ligand is covalently attached to the drug via a first linker (L1) and wherein the second ligand is covalently attached to the drug via a second linker (L2).

6. The construct of claim 2, wherein the drug is covalently joined to the ligand and the molecular weight increasing moiety through a linker (L3).

7. The construct of claim 2, wherein the drug is covalently joined to the ligand and the molecular weight increasing moiety through a first linker (L1) a second linker (L2), respectively.

8. The construct of claim 2, wherein the molecular weight increasing moiety has a molecular weight greater than 10,000 Da.

9. The construct of claim 1, wherein the first and second binding partners are the same molecular species.

10. The construct of claim 1, wherein the drug is further covalently joined to a third ligand having affinity for a third binding partner.

11. The construct of claim 1, wherein the Kd of the construct is not more than about 10 times the Kd of the free drug.

12. The construct of claim 2, wherein the Kd of the construct is not more than about 10 times the Kd of the free drug.

13. A method of modifying pharmacokinetics of a drug comprising the step of: incorporating the drug into a construct according to claim 1.

14. A method of modifying pharmacokinetics of a drug comprising the step of: incorporating the drug into a construct according to claim 2.

15. A construct comprising a drug covalently joined to a ligand having affinity for a binding partner of a physiological fluid, wherein in the fluid an equilibrium is formed between (a) the construct in free form which retains relatively more of the drug's activity, and (b) the construct in a binary complex with the binding partner, which retains relatively less of the drug's activity, wherein the drug has a molecular weight of at least 10,000 Da.
